(19) United States
(12) Patent Application Publication
(10) Pub. No.: US 2008/0287869 A1
(43) Pub. Date: Nov. 20, 2008

(54) Safety Syringe

(76) Inventor: Thomas Chun, Northridge, CA (US)

Correspondence Address: ITALIA IP, 3500 WEST OLIVE AVE., SUITE 300, BURBANK, CA, 91505, US

(21) Appl. No.: 11/750,327

(22) Filed: May 17, 2007

(51) Int. Cl. A61M 5/00; A61M 5/32

(52) U.S. Cl. 604/110; 604/195; 604/196

(57) ABSTRACT

A safety syringe that includes syringe assembly retained within a hollow tubular shell, whereby a retaining member is utilized for retaining the syringe assembly in an operative position such that it is available for administering a shot. After the safety syringe is used, a retracting assembly retracts the syringe assembly into the shell and locks it in place thereby preventing any accidental pricks.

### BACKGROUND

This application relates generally to an automatically retractable safety syringe. More specifically, this application relates to a syringe designed to have a needle assembly that automatically retracts and locks into an inoperative position at the completion of its use to administer a given dose.

### SUMMARY

This application discloses a safety syringe that is economical to produce, of simple construction and capable of mass production.

In particular, this application discloses a safety syringe comprising: a hollow tubular shell comprising an elongated cylinder having a longitudinal bore therethrough of decreasing diameter, wherein the upper seating portion has the largest diameter, a mid-portion has a mid diameter, a lower portion with a smaller diameter, and a lower opening with the smallest diameter; a syringe assembly for slideably inserting into said shell, wherein the syringe assembly comprises a hollow tubular barrel for slideably receiving a plunger, wherein the barrel further comprises a flared portion at the plunger receiving end and a needle attachment portion for attachment of a needle assembly at the other end, and wherein the plunger includes a head portion for a user to depress when administering a shot; a retaining member fixably seated in said upper seating portion for retaining said syringe assembly in an operative position within said shell prior to use, wherein the retaining member includes a releasing portion and spring attachment portion; a retracting assembly for retracting said needle assembly through said lower opening and into said shell after use, said retracting assembly comprising a spring attached to said spring attachment portion of said retaining member at one end and further extending to surround said barrel of said syringe assembly, wherein said spring is retained at the other end at the needle attachment portion of said barrel, and wherein in the operative position prior to use, the flared portion of the barrel is retained by the releasing portion of the retaining member such that the spring is loaded when in the operative position and whereby when the plunger is substantially fully depressed, the head portion engages the releasing portion of the retaining member and thereby releases the flared portion of the barrel and the spring contracts and retracts the needle assembly into the shell through the lower opening; and, locking means for preventing said needle assembly from exiting said lower opening after use.

In another embodiment, this application discloses a safety syringe comprising: a hollow tubular shell comprising an elongated cylinder having a longitudinal bore therethrough of decreasing diameter, wherein the upper seating portion has the largest diameter, a mid-portion has a mid diameter, a lower portion with a smaller diameter, and a lower opening with the smallest diameter; a syringe assembly for slideably inserting into said shell, wherein the syringe assembly comprises a hollow tubular barrel for slideably receiving a plunger, wherein the barrel further comprises a flared portion at the plunger receiving end and a needle attachment portion for attachment of a needle assembly at the other end, and wherein the plunger includes a head portion for a user to depress when administering a shot; a retaining member fixably seated in said upper seating portion for retaining said syringe assembly in an operative position within said shell prior to use, wherein the retaining member includes a releasing portion comprising at least one deflectable flange and spring attachment portion; a retracting assembly for retracting said needle assembly through said lower opening and into said shell after use, said retracting assembly comprising a spring attached to said spring attachment portion of said retaining member at one end and further extending to surround said barrel of said syringe assembly, wherein said spring is retained at the other end at the needle attachment portion of said barrel, and wherein in the operative position prior to use, the flared portion of the barrel is retained by the releasing portion of the retaining member such that the spring is loaded when in the operative position and whereby when the plunger is substantially fully depressed, the head portion engages the releasing portion of the retaining member and thereby releases the flared portion of the barrel and the spring contracts and retracts the needle assembly into the shell through the lower opening; locking means for preventing said needle assembly from exiting said lower opening after use; and, solute containing means attached at a position between the needle attachment portion and the needle assembly.

In yet a further embodiment, this application discloses a safety syringe comprising: a hollow tubular shell comprising an elongated cylinder having a longitudinal bore therethrough of decreasing diameter, wherein the upper seating portion has the largest diameter, a mid-portion has a mid diameter, a lower portion with a smaller diameter, and a lower opening with the smallest diameter; a syringe assembly for slideably inserting into said shell, wherein the syringe assembly comprises a hollow tubular barrel for slideably receiving a plunger, wherein the barrel further comprises a flared portion at the plunger receiving end and a needle attachment portion for attachment of a needle assembly at the other end, and wherein the plunger includes a head portion for a user to depress when administering a shot; a retaining member fixably seated in said upper seating portion for retaining said syringe assembly in an operative position within said shell prior to use, wherein the retaining member includes a releasing portion comprising at least one deflectable flange and spring attachment portion, wherein the spring attachment portion of the retaining member is a ribbed portion protruding around the outer circumference of the retaining member; a retracting assembly for retracting said needle assembly through said lower opening and into said shell after use, said retracting assembly comprising a spring attached to said spring attachment portion of said retaining member at one end and further extending to surround said barrel of said syringe assembly, wherein said spring is retained at the other end at the needle attachment portion of said barrel, and wherein in the operative position prior to use, the flared portion of the barrel is retained by the releasing portion of the retaining member such that the spring is loaded when in the operative position and whereby when the plunger is substantially fully depressed, the head portion engages the releasing portion of the retaining member and thereby releases the flared portion of the barrel and the spring contracts and retracts the needle assembly into the shell through the lower opening; locking means for preventing said needle assembly from exiting said lower opening after use; and, solute containing means attached at a position between the needle attachment portion and the needle assembly.

### BRIEF DESCRIPTION OF THE DRAWINGS

The drawings, when considered in connection with the following description, are presented for the purpose of facilitating an understanding of the subject matter sought to be protected.

FIG. 1 is a front elevational view of a first embodiment of the safety syringe described herein shown prior to use;

FIG. 2 is a sectional view of the safety syringe taken generally along the line 2-2 in FIG. 1;

FIG. 3 is a front elevational view of the first embodiment of the safety syringe shown in FIG. 1 shown after use;

FIG. 4 is a sectional view of the safety syringe taken generally along the line 4-4 in FIG. 3;

FIG. 5 is a front elevational view of a second embodiment of the safety syringe described herein shown after use;

FIG. 6 is a sectional view of the safety syringe taken generally along the line 6-6 in FIG.5;

FIG. 7 is an exploded view of the safety syringe of FIG. 1;

FIG. 8 is a front elevational view of the retaining member;

FIG. 9 is a sectional view of the retaining member taken generally along the line 9-9 in FIG.8;

FIG. 10 is a front elevational view of the locking clip; and, FIG. 11 is a side elevational view of the locking clip in FIG. 10.

### DETAILED DESCRIPTION

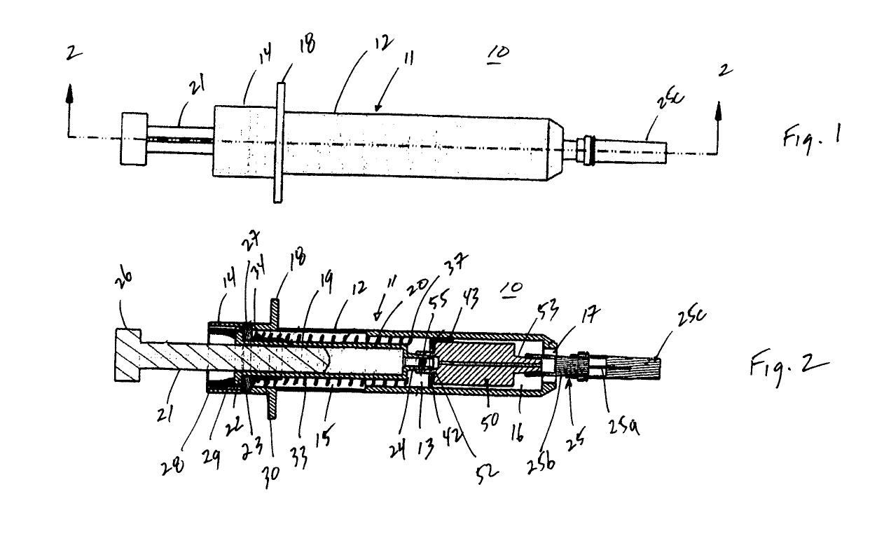

Referring to FIGS. 1-4, shown therein and generally designated by the reference character 10 is a safety syringe constructed in accordance with the following description. The safety syringe 10 includes a hollow tubular shell 11 comprising an elongated cylinder 12 having a longitudinal bore 13 therethrough of decreasing diameter, wherein an upper seating portion 14 has the largest diameter, a mid-portion 15 has a mid diameter, a lower portion 16 with a smaller diameter, and a lower opening 17 with the smallest diameter. The syringe also includes a substantially circular grip 18 integral with the surface of the shell 11. The syringe 10 further includes a syringe assembly 19 for slideably inserting into said shell 11, wherein the syringe assembly 19 comprises a hollow tubular barrel 20 for slideably receiving a plunger 21, wherein the barrel 20 further comprises a flared portion 22 at the plunger receiving end 23 and a needle attachment portion 24 for attachment of a needle assembly 25 at the other end. The needle assembly is common in the art and includes the needle 25a integral to an attachment member 25b. The needle assembly may also include a cap 25c for fitment upon the needle 25a prior to use.

The plunger 21 further includes a head portion 26 for a user to depress when administering a shot. The substantially circular grip 18 is provided to be gripped with the index and middle fingers of the user while applying pressure to the plunger head portion 26 with the thumb of the of the same hand during the injection stroke.

A retaining member 27 is fixably seated using either frictional fit or any other attachment means as is common in the art in said upper seating portion 14 for retaining the syringe assembly 19 in an operative position within said shell 11 prior to use. As used herein, the term “operative position” refers to the position depicted in FIGS. 1 and 2, whereby the needle assembly 25 is located outside the shell 11 and the plunger 21 is partially retained within the barrel 20. As shown more clearly in FIGS. 8 and 9, the retaining member 27 includes a releasing portion 28 comprising a pair of deflectable flanges 29 which taper in toward the centerline of the longitudinal bore 40 of the retaining member 27. It should be appreciated that although two flanges 29 are pictured in the embodiment described, a single flange would also be effective. The retaining member 27 also includes an integral spring attachment portion 30, wherein the spring attachment portion 30 of the retaining member 27 is a single ribbed portion 31 protruding around the outer circumference 32 of the retaining member 27. A spring 33 is retained upon the retaining member 27 and more specifically, the ribbed portion 31 of the spring attachment portion 30, by butting one end 34 of the spring against the ribbed portion 31. This may be accomplished by screwing the one end 34 of the spring 33 onto the surface of the spring attachment portion 30 such that the end 34 of the spring 33 is retained on the opposite side 35 of the ribbed portion 31 relative to the rest of the spring 33. See also FIG. 2.

The safety syringe also includes a retracting assembly 36 for retracting the needle assembly 25 through the lower opening 16 and into the shell 11 after use. The retracting assembly, as partially described above, includes the spring 33 attached to the spring attachment portion 30 of the retaining member 27 wherein the spring 33 further extends to surround the barrel 20 of the syringe assembly 19. The opposite end 37 of the spring 33 is retained upon the needle attachment portion 24 of the barrel 20 by way of the spring having a smaller diameter than the barrel 30 such that the opposite end 37 of the spring 33 maintains in contact with the first vertical surface 38 of the barrel 30 adjacent to the needle attachment portion 24. Thus, when the safety syringe is in an operative position prior to use, the flared portion 22 of the barrel is retained by the releasing portion of the retaining member (under the flared portions 29) such that the spring 33 is loaded when in the operative position. As used herein, the term “loaded” refers to when a force created within the spring after an initial force is applied to the spring 33 to lengthen it. More specifically, a recoiling force is created in the spring that will act to force the spring back to its non-lengthened state. The load is created in the spring while in the operative position by inserting the needle attachment portion 24 of the barrel 20 through the longitudinal bore 40 of the retaining member 27.

As described above, the retaining member is fixably attached to the shell in the upper seating position 14. Once through the retaining member the barrel 20 continues on to pass through the spring 33 attached to the spring attachment portion 30 of the retaining member 27 until the vertical surface 38 of the barrel 20 engages the end 37 of the spring 33. At this stage a force is required to overcome the spring force in the opposite direction. The user continues to insert the barrel 30 into the spring 33, retaining member 27 and shell 11 such that the spring 33 is lengthened. This continues until the flared portion 22 of the barrel engages the flanged portions 29 of the releasing portion 28 of the retaining member 27 such that the flanges 29 deflect in a direction outward from the centerline of the longitudinal bore 40. Once the barrel is pushed just a little further, the flared portion 22 of barrel 20 is seated at a position 41 below the flanges 29 which then deflect in a direction toward the centerline of the longitudinal bore 40 and above a portion of the flared portion 22 such that the barrel 20, and more specifically, the spring 33 is maintained in a loaded position. See FIG. 7.

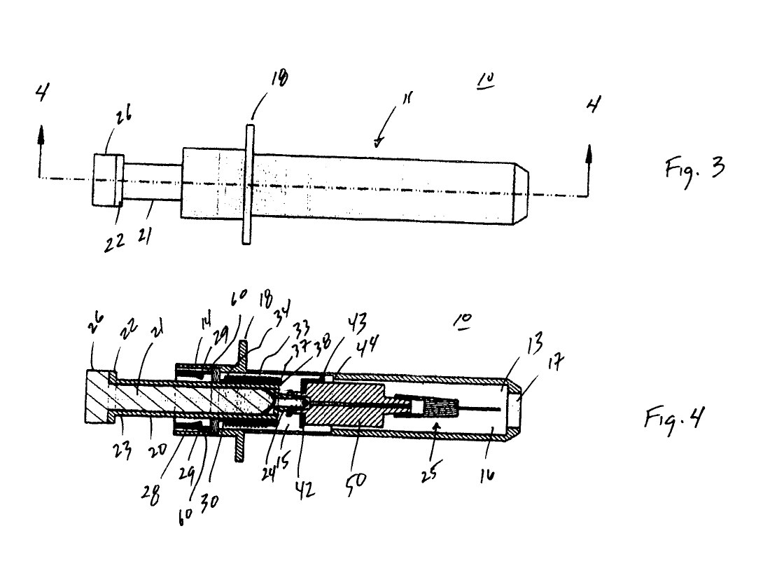

Referring now to FIGS. 3 and 4, shown generally therein is the safety syringe 10 after use which is created when the plunger is substantially fully depressed. As used herein the term “substantially fully depressed” refers to when the plunger head portion 26 is depressed sufficiently into the barrel 20 such that it engages the releasing portion 28 of the retaining member 27 and thereby releases the flared portion 22 of the barrel 20 and the spring 33 contracts and retracts the needle assembly 25 into the shell 11 through the lower opening 17. More specifically, during the retracting phase, once the plunger head portion 26 is substantially fully depressed, the flanges 29 of the retaining member 27 are again deflected in a direction opposite the centerline of the longitudinal bore 40 and the surfaces 60 previously retaining the barrel 20 and the spring 33 in the loaded position are cleared and the syringe assembly is forced in and upward direction due to the contraction of the spring 33, thereby retracting the attached needle assembly 25 into the shell 11 through the lower opening 17.

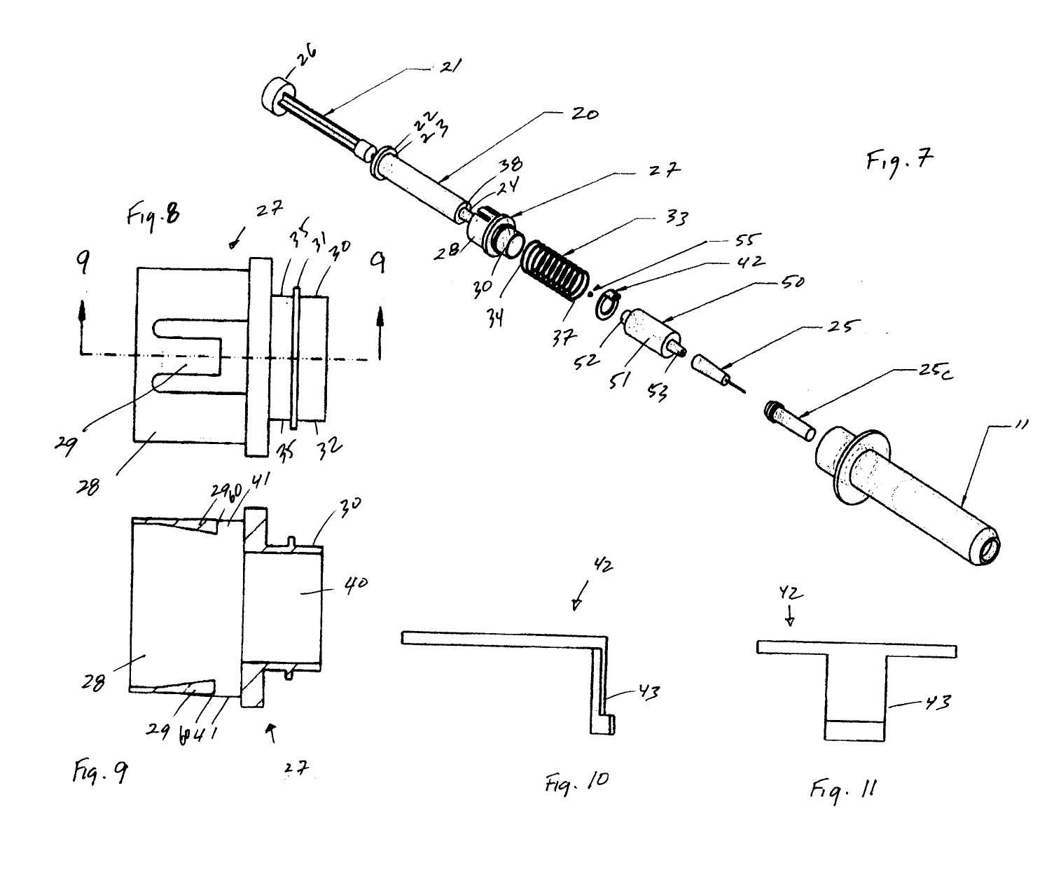

Referring now to FIGS. 4, 10 and 11, the safety syringe includes a locking means for preventing said needle assembly 25 from exiting said lower opening 13 after use. The locking means is a locking clip 42 fixably attached to the needle attachment portion 24 of the barrel 20 by means common in the art and includes at least one peg portion 43 for catching the a lower portion 16 of the shell 11. When the safety syringe is in the operative position the peg portion 43 is flexed inward in a direction toward the centerline of the longitudinal bore 13 based on its contact with the inner surface of the lower portion 16. However, once the safety syringe 10 is used and the retraction of the needle assembly 25 is activated, the locking 42 clip is forced in an upward direction into the mid-portion 15 of a larger diameter such that it clears the inner surface of the lower portion 16. When located in the mid-portion 15 (the after use position), the peg portion 43 deflects in a direction opposite the centerline of the longitudinal bore 13 such that when the plunger 21 is depressed once again, the peg portion catches the surface 44 located at a point between the mid-portion 15 and the lower portion 16, thereby restricting the needle assembly 25 from exiting through the lower opening 17. See FIG. 4.

Further, as indicated in FIGS. 1-4, and 7, the safety syringe may include a solute containing means such as a solute container 50 attached at a position between the needle attachment portion 24 and the needle assembly. The solute container 50 is utilized when the safety syringe is provided with a liquid solvent in the barrel 20 and a dry solute in the container 50 such that when the shot is administered, the solvent is combined with the solute such that an effective dose of the solute is administered by the user. The solute container 50 includes a substantially hollow main body 51 for holding the solute and a barrel attachment portion 52 at one end and a needle assembly attachment portion 53 at the other. The solute container 50 may also include means for providing that the solute is effectively solubilzed when the solvent is introduced.

The safety syringe 10 may also include blocking means for preventing solvent retained within the barrel 20 from entering the solute container 50 prior to use. For example, the blocking means may be a ball 55 that is sized to be frictionally retained within said needle attachment portion 24 such that when sufficient force is applied to the plunger head 26, the ball 55 is expelled from the needle attachment portion 24 and the solvent is able to enter the solvent container 50.

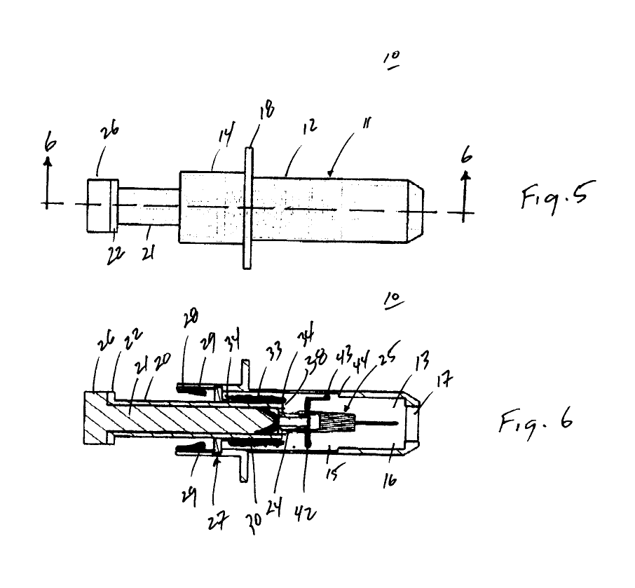

Finally, as shown in FIGS. 5 and 6, a second embodiment of the safety syringe 10 is shown wherein the solvent container 50 is not used, but the functionality and operation is the same as described above.

In the construction of the safety syringe 10, a majority of the components may be formed of suitable plastics that may be molded, however, components that must withstand load bearing and other forces such as the spring 33 and the needle 25a may be formed of suitable metals.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A safety syringe comprising:
a hollow tubular shell comprising an elongated cylinder having a longitudinal bore therethrough of decreasing diameter, wherein an upper seating portion has the largest diameter, a mid-portion has a mid diameter, a lower portion with a smaller diameter, and a lower opening with the smallest diameter;
a syringe assembly for slideably inserting into said shell, wherein the syringe assembly comprises a hollow tubular barrel for slideably receiving a plunger, wherein the barrel further comprises a flared portion at the plunger receiving end and a needle attachment portion for attachment of a needle assembly at the other end, and wherein the plunger includes a head portion for a user to depress when administering a shot;
a retaining member fixably seated in said upper seating portion for retaining said syringe assembly in an operative position within said shell prior to use, wherein the retaining member includes a releasing portion and spring attachment portion;
a retracting assembly for retracting said needle assembly through said lower opening and into said shell after use, said retracting assembly comprising a spring attached to said spring attachment portion of said retaining member at one end and further extending to surround said barrel of said syringe assembly, wherein said spring is retained at the other end at the needle attachment portion of said barrel, and wherein in the operative position prior to use, the flared portion of the barrel is retained by the releasing portion of the retaining member such that the spring is loaded when in the operative position and whereby when the plunger is substantially fully depressed, the head portion engages the releasing portion of the retaining member and thereby releases the flared portion of the barrel and the spring contracts and retracts the needle assembly into the shell through the lower opening; and,
locking means for preventing said needle assembly from exiting said lower opening after use.

2. The safety syringe of claim 1 further comprising a solute container attached at a position between the needle attachment portion and the needle assembly.

3. The safety syringe of claim 2 wherein the needle attachment portion includes blocking means for preventing solvent retained within said barrel from entering said solute container prior to use.

4. The safety syringe of claim 3 wherein the blocking means is a ball that is sized to be frictionally retained within said needle attachment portion; such that when sufficient force is applied to the plunger head, the ball is expelled from the needle attachment portion and the solvent is able to enter the solvent container.

5. The safety syringe of claim 1 wherein the locking means is a locking clip fixably attached to said needle attachment portion of said barrel and includes at least one peg portion for catching the a lower portion or the shell.

6. The safety syringe of claim 5 further comprising a solute container attached at a position between the needle attachment portion and the needle assembly.

7. The safety syringe of claim 6 wherein the needle attachment portion includes blocking means for preventing solvent retained within said barrel from entering said solute container prior to use.

8. The safety syringe of claim 7 wherein the blocking means is a ball that is sized to be frictionally retained within said needle attachment portion; such that when sufficient force is applied to the plunger head, the ball is expelled from the needle attachment portion and the solvent is able to enter the solvent container.

9. The safety syringe of claim 1 wherein the releasing portion of the retaining member comprises at least one deflectable flange that deflects inward to retain the flared portion of the barrel when the safety syringe is in the operative position and deflects outward by the engagement of the plunger head when the plunger is substantially fully depressed.

10. The safety syringe of claim 1 wherein the spring attachment portion of the retaining member is a ribbed portion protruding around the outer circumference of the retaining member.

11. The apparatus of claim 10 wherein a spring is attached to the spring attachment portion by butting one end of the spring against the ribbed portion.

12. A safety syringe comprising:
a hollow tubular shell comprising an elongated cylinder having a longitudinal bore therethrough of decreasing diameter, wherein an upper seating portion has the largest diameter, a mid-portion has a mid diameter, a lower portion with a smaller diameter, and a lower opening with the smallest diameter;
a syringe assembly for slideably inserting into said shell, wherein the syringe assembly comprises a hollow tubular barrel for slideably receiving a plunger, wherein the barrel further comprises a flared portion at the plunger receiving end and a needle attachment portion for attachment of a needle assembly at the other end, and wherein the plunger includes a head portion for a user to depress when administering a shot;
a retaining member fixably seated in said upper seating portion for retaining said syringe assembly in an operative position within said shell prior to use, wherein the retaining member includes a releasing portion comprising at least one deflectable flange and spring attachment portion;
a retracting assembly for retracting said needle assembly through said lower opening and into said shell after use, said retracting assembly comprising a spring attached to said spring attachment portion of said retaining member at one end and further extending to surround said barrel of said syringe assembly, wherein said spring is retained at the other end at the needle attachment portion of said barrel, and wherein in the operative position prior to use, the flared portion of the barrel is retained by the releasing portion of the retaining member such that the spring is loaded when in the operative position and whereby when the plunger is substantially fully depressed, the head portion engages the releasing portion of the retaining member and thereby releases the flared portion of the barrel and the spring contracts and retracts the needle assembly into the shell through the lower opening;
locking means for preventing said needle assembly from exiting said lower opening after use; and,
solute containing means attached at a position between the needle attachment portion and the needle assembly.

13. The safety syringe of claim 12 wherein the solute containing means is a solute container.

14. The safety syringe of claim 13 wherein the needle attachment portion includes blocking means for preventing solvent retained within said barrel from entering said solute container prior to use.

15. The safety syringe of claim 14 wherein the blocking means is a ball that is sized to be frictionally retained within said needle attachment portion; such that when sufficient force is applied to the plunger head, the ball is expelled from the needle attachment portion and the solvent is able to enter the solvent container.

16. A safety syringe comprising:
a hollow tubular shell comprising an elongated cylinder having a longitudinal bore therethrough of decreasing diameter, wherein an upper seating portion has the largest diameter, a mid-portion has a mid diameter, a lower portion with a smaller diameter, and a lower opening with the smallest diameter;
a syringe assembly for slideably inserting into said shell, wherein the syringe assembly comprises a hollow tubular barrel for slideably receiving a plunger, wherein the barrel further comprises a flared portion at the plunger receiving end and a needle attachment portion for attachment of a needle assembly at the other end, and wherein the plunger includes a head portion for a user to depress when administering a shot;
a retaining member fixably seated in said upper seating portion for retaining said syringe assembly in an operative position within said shell prior to use, wherein the retaining member includes a releasing portion comprising at least one deflectable flange and spring attachment portion, wherein the spring attachment portion of the retaining member is a ribbed portion protruding around the outer circumference of the retaining member;
a retracting assembly for retracting said needle assembly through said lower opening and into said shell after use, said retracting assembly comprising a spring attached to said spring attachment portion of said retaining member at one end and further extending to surround said barrel of said syringe assembly, wherein said spring is retained at the other end at the needle attachment portion of said barrel, and wherein in the operative position prior to use, the flared portion of the barrel is retained by the releasing portion of the retaining member such that the spring is loaded when in the operative position and whereby when the plunger is substantially fully depressed, the head portion engages the releasing portion of the retaining member and thereby releases the flared portion of the barrel and the spring contracts and retracts the needle assembly into the shell through the lower opening;
locking means for preventing said needle assembly from exiting said lower opening after use; and, solute containing means attached at a position between the needle attachment portion and the needle assembly.

17. The safety syringe of claim 16 wherein the solute containing means is a solute container.

18. The safety syringe of claim 17 wherein the needle attachment portion includes blocking means for preventing solvent retained within said barrel from entering said solute container prior to use.

19. The safety syringe of claim 18 wherein the blocking means is a ball that is sized to be frictionally retained within said needle attachment portion; such that when sufficient force is applied to the plunger head, the ball is expelled from the needle attachment portion and the solvent is able to enter the solvent container.

20. The safety syringe of claim 16 wherein the locking means is a locking clip fixably attached to said needle attachment portion of said barrel and includes at least one peg portion for catching the a lower portion or the shell.
